(19) United States
(12) Patent Application Publication
(10) Pub. No.: US 2025/0114650 A1
(43) Pub. Date: Apr. 10, 2025

(54) A PLATFORM FOR RESPIRATORY PROTECTIVE EQUIPMENT

(71) Applicant: IMPERTECH INDUSTRIES Ltd., Barkan (IL)

(72) Inventor: Israel GURMAN, Rehovot (IL)

(21) Appl. No.: 18/726,332

(22) PCT Filed: Dec. 27, 2022
(86) PCT No.: PCT/IL2022/051394

§ 371 Date: Jul. 2, 2024

(30) Foreign Application Priority Data
Jan. 2, 2022 (IL) 289562

(51) Int. Cl. A62B 18/08; A61M 16/06; A62B 7/10; A62B 18/02; A62B 18/10; A62B 23/02

(52) U.S. Cl. CPC A62B 18/08; A61M 16/0616; A62B 7/10; A62B 18/02; A62B 18/086; A62B 18/10; A62B 23/02

(57) ABSTRACT

An integrative interface platform for respiratory protective equipment, comprising a main body which is configured with at least five interfaces, with each of which a corresponding ancillary device, including an air inlet, is in fluid or sound communication with a confined space between the respiratory protective equipment and a face of a wearer, when the respiratory protective equipment is worn on a face, is sealingly engaged, wherein second and third interfaces which are superiorly and inferiorly spaced, respectively, from a first interface are also angularly spaced in opposite directions from the first interface and the ancillary device of a fourth interface is posteriorly spaced from the ancillary device of the first interface.

### FIELD OF THE INVENTION

The present invention is in the field of respiratory protection equipment. More specifically, the invention relates to a platform, and particularly an integrative interface platform, for respiratory protective equipment.

### BACKGROUND OF THE INVENTION

The primary objective of respiratory protective equipment is to enable its wearers to unrestrictedly breathe air, free of airborne noxious agents, while performing their intended tasks.

One implementation of the abovementioned respiratory protective equipment is respiratory protective masks, commonly used personnel of security forces and other organizations that are intended to operate at possible contaminated areas. Those respiratory protective masks are generally provided with a flexible tight-fitting face-piece of uniform thickness, such as to form a protected confined space around a wearer's face, where one or more air inlets/outlets are embedded within the tight-fitting covering through which filtered air is inhaled and exhaled air rich in carbon-dioxide is evacuated.

During the decades of use of respiratory protective masks, various types of ancillary devices were added to the basic mask, such as drinking aids and radio communication means. However, each of the added ancillary devices communicates with the protected confined space while crossing through the tight-fitting face-piece, thus compromising the air-tightness and the respiratory protection provided by the mask. Furthermore, each added enhancement requires a further set of components and at least one face-piece aperture, thus producing a more complex and expensive to manufacture face-piece mold, as well as a heavier and more cumbersome mask.

It is therefore an object of the present invention to provide an integrative interface platform for respiratory protective equipment that enables use of the abovementioned enhancements without compromising air-tightness.

It is another object of the present invention to provide an integrative interface platform of increased compactness relative to prior art practice and of a minimal number of parts.

It is another object of the present invention to provide an integrative interface platform that is easily and quickly removable from respiratory protective equipment upon demand.

Other objects and advantages of the invention will become apparent as the description proceeds.

### SUMMARY OF THE INVENTION

An integrative interface platform for respiratory protective equipment comprises a main body which is configured with at least five interfaces, with each of which a corresponding ancillary device, including an air inlet, is in fluid or sound communication with a confined space between said respiratory protective equipment and a face of a wearer, when said respiratory protective equipment is worn on a face, is sealingly engaged, wherein second and third interfaces which are superiorly and inferiorly spaced, respectively, from a first interface are also angularly spaced in opposite directions from the first interface and the ancillary device of a fourth interface is posteriorly spaced from the ancillary device of the first interface.

In one embodiment, the main body comprises five interfaces and each of the corresponding ancillary devices is selected from one or more of the group of:
a) an air outlet assembly, positioned at an inferior portion of the platform, which comprises a one-way valve for enabling evacuation of humid exhaled air containing CO2 via an air outlet during exhalation;
b) an air inlet assembly, which is located at an anterior region of the platform and in communication with a one-way valve for facilitating an influx of air via the air inlet during inhalation;
c) a communication device adapted to connect with external communication equipment;
d) a speech device, for enabling transmission therethrough of voice; and
e) an adjustable drinking implement.

In one aspect, the air outlet assembly is also configured to facilitate discharge of liquid that has accumulated at the inferior portion.

In one aspect, the air inlet is adapted to receive an outlet of a filtered air supply source.

In one aspect, the adjustable drinking implement is an adjustable drinking straw, comprising an adjustment lever which is positionable at a stored state or a deployed state, wherein said adjustable drinking straw connects with an inlet tube having a drinking vessel connector for enabling drinking therefrom.

In one aspect, the first interface is the air inlet, the second interface is an interface for the speech device, the third interface is the air outlet, the fourth interface is an interface for the communication device, and a fifth interface is an interface for the adjustable drinking implement.

Respiratory protective equipment comprises the integrative interface platform.

The platform is preferably sealingly engaged within an aperture formed in the respiratory protective equipment, such that the platform is disengageable from the respiratory protective equipment.

In one aspect, the respiratory protective equipment further comprises an air-tight coupling between the platform and the respiratory protective equipment for maintaining sealed engagement therebetween.

In one aspect, the air-tight coupling comprises a clamping tie that is engageable around an annular lip surrounding the aperture formed in the respiratory protective equipment and that is tightened by a fastening lock.

The platform may comprise a handle by which a manual force that causes a posterior edge of the main body or of a protrusion thereof to forcefully press on a sealing element provided with a surrounding edge of the aperture formed in the respiratory protective equipment and to be sealingly engaged with the surrounding edge is transmittable.

In one aspect, the respiratory protective equipment is a mask, and may comprise one or more lateral air inlets, each of which is adapted to receive an outlet of a filtered air supply source.

In one aspect, the respiratory protective equipment further comprises a hollow nose-mouth protective cup which is coupled to the platform while each of the corresponding ancillary devices remains in communication with the confined space. The nose-mouth cup is preferably configured with a plurality of air guiding surfaces which protrude from an outer wall of the nose-mouth cup to guide exhaled air through the air outlet of the platform.

### BRIEF DESCRIPTION OF THE DRAWINGS

The above and other characteristics and advantages of the invention will be better understood through the following illustrative and non-limitative detailed description of preferred embodiments thereof, with reference to the appended drawings, wherein:

FIG. 1 is an exploded view of an embodiment of a respiratory protective mask configured with an integrative interface platform;

FIG. 2A is a perspective anterior view from above of an embodiment of an integrated interface platform, shown without a protective cover to expose the integrated interfaces;

FIG. 2B is another perspective proximal view of the integrated interface platform of FIG. 2A, shown with the protective cover;

FIG. 2C is a cross sectional view of the integrated interface platform, cut along plane A-A of FIG. 2B;

FIGS. 3A and 3B is a perspective posterior view of the integrated interface platform of FIG. 2A, shown with the protective cover and illustrating stored and deployed states, respectively, of a drinking straw;

FIG. 4 is a perspective view of an embodiment of a respiratory protective mask configured with an integrated interfaces platform and a filter;

FIG. 5A is an embodiment of a respiratory protective mask configured with an integrated interface platform and a nose-mouth cup, the respiratory protective mask being shown in a transparent condition; and

FIG. 5B is a perspective posterior view of the nose-mouth cup of FIG. 5A when coupled to the integrated interface platform, shown without the respiratory protective mask.

### DETAILED DESCRIPTION OF THE INVENTION

The present invention relates to an integrative interface platform configured with multiple interfaces for respiratory protective equipment ancillary devices, such as a drinking device, a speech membrane, air inlet, air outlet, and communication device. The platform may be dedicated for use with specific types of interfaces or ancillary devices, and may be removed from the respiratory protective equipment in order to be cleaned or replaced with another platform configured with other types of interfaces or ancillary devices.

The integrated interfaces are located and oriented to optimally utilize the limited confined space between the respiratory mask and the wearer's face, as well as to avoid any interference during an operation involving one or more of the interfaces. When the integrative platform comprises for example at least four interfaces or at least five interfaces, although a compactly configured platform may also comprise less than four interfaces, the limited confined space of the platform is compactly and efficiently utilized when second and third interfaces which are superiorly and inferiorly spaced, respectively, from a first interface are also angularly spaced in opposite directions from the first interface and a fourth interface is posteriorly spaced from the first interface.

In the following detailed description, references are made to several embodiments of the present invention, examples of which are illustrated in the accompanying figures. The figures depict embodiment of the present invention for purposes of illustration only. One skilled in the art will readily recognize from the following description that the described embodiments may be combined, alternative embodiments may be utilized, and other changes may be made without departing from the spirit or scope of the present invention described herein.

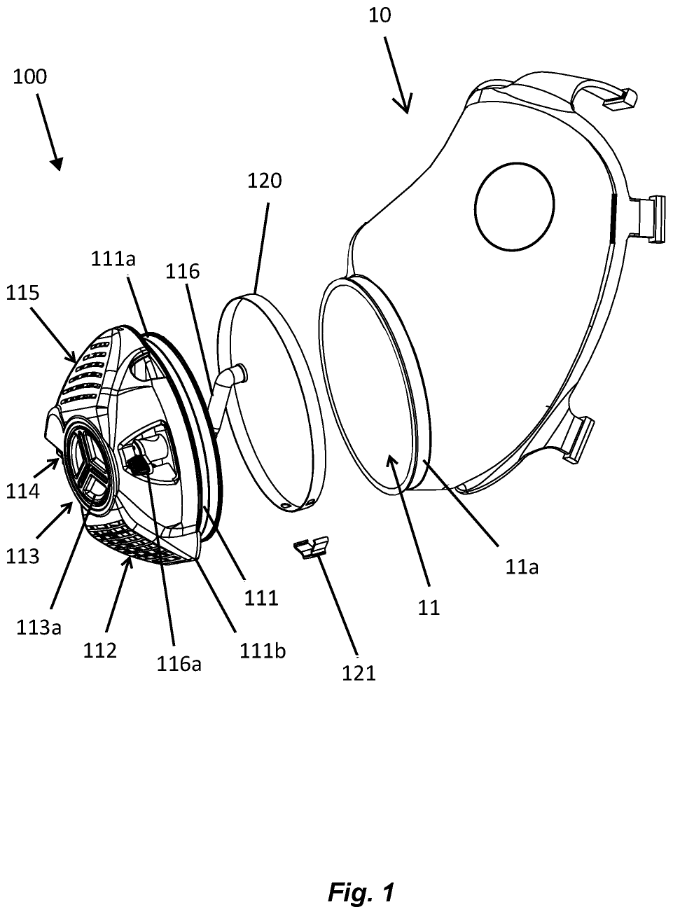

FIG. 1 schematically illustrates an exploded view of respiratory protective mask 10 adapted to be coupled with an integrative interface platform 100, according to an embodiment of the present invention. Mask 10 is a flexible tight-fitting face-piece of uniform thickness, generally made of elastomeric material, and having an interface aperture 11 delimited by an annular clamping lip 11a, which is adapted to receive a rigid clamping protrusion 111a of platform 100. Protrusion 111a is threadedly engageable within clamping lip 11a, and clamping tie 120 is threadedly engageable around clamping lip 11a and tightened by a fastening lock 121, in order to firmly clamp lip 11a against the rigid clamping protrusion 111a of platform 100, thereby providing an air-tight coupling between platform 100 and mask 10. Other types of an air-tight coupling between platform 100 and mask 10 are also within the scope of the invention.

The posterior periphery of the main body 111 of platform 100, or protrusion 111a thereof, which is preferably round, for example oval, but may be shaped in other ways as well, may also be frictionally and sealingly engageable with an edge surrounding aperture 11. Of course the edge surrounding aperture 11 will be resealed with the periphery of platform 100, or of a platform replacing platform 100, following removal of the platform and the subsequent coupling of a desired platform with mask 10.

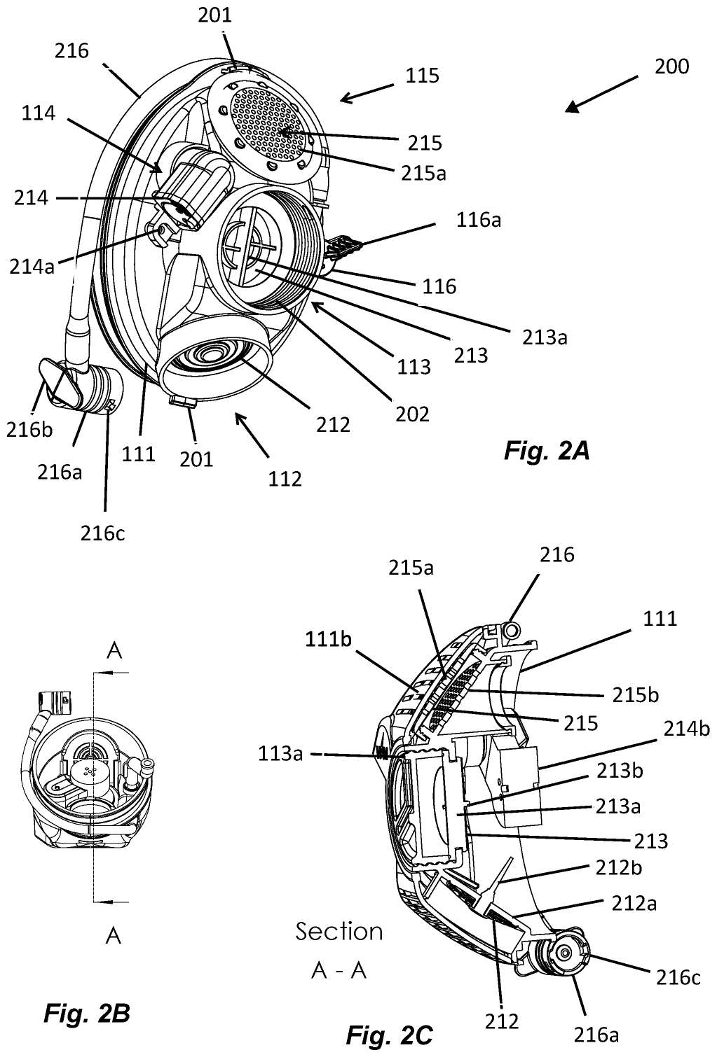

The main body 111 of platform 100 may be made of a rigid material, and comprises air-tight interfaces for an air outlet 112, an air inlet 113 (i.e., which may be covered by lid 113a when not in use), a communication device 114, a speech device 115, and an adjustable drinking straw 116 (i.e., by an adjustment lever 116a). Interfaces 112-116 are generally externally protected by a cover 111b having corresponding apertures, and are further illustrated in FIGS. 2A-2B.

Speech device 115 is superiorly spaced from air inlet 113, and air outlet 112 is inferiorly spaced from air inlet 113. Both air outlet 112 and speech device 115 are angularly spaced from air inlet 113, for example by an angle ranging from 40-50 degrees, generally in opposite angular positions, and may be symmetrically positioned. Cover 111b enclosing these interfaces is also similarly configured. The interface of communication device 114 may be laterally spaced from air inlet 113.

FIG. 2A illustrates an integrated interface platform 200 with cover 111b and air inlet lid 113a removed, according to an embodiment of the invention, thus exposing interfaces 112-116, and FIG. 2C illustrates a center section view of a fully assembled platform 200 (i.e., with cover 111b attached thereto).

Air outlet 112 and air inlet 113 of platform 200 utilize one-way valves 212 and 213 correspondingly, for urging air flow in a desired direction. The axial displacement of one-way valve 212 within its chamber, connected to main body 111, is posteriorly limited by ribs 212a which are formed with air grooves, to allow air flow therethrough only during exhalation, thus inducing outward pressure on valve 212. Conversely, the axial displacement of one-way valve 213 is limited anteriorly by ribs 213a which are formed with air grooves in communication with environmental air to allow air flow therethrough only during inhalation, thus inducing an inward pressure on valve 213. An air filter, which may be conventional, may be threaded in lieu of lid 113a. Valves 212 and 213 may be formed of thin and flexible elastic annular discs which are centrally fixed to main body 111 by detachable or fixed fastening elements 212b and 213b, respectively.

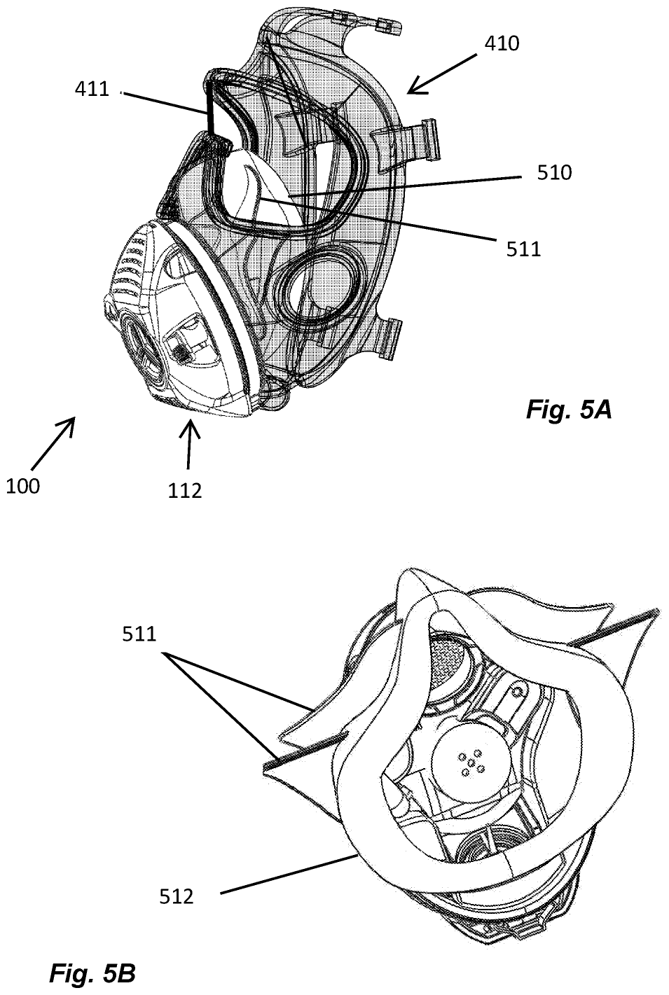

Air outlet 112 is positioned at the anterior and inferior portion of platform 200 in order to facilitate smooth evacuation of exhaled, relatively heavy air containing a high humidity content and CO2, while air inlet 113 is positioned centrally with respect to platform 200 and anteriorly to the estimated mouth and nose position of the wearer of mask 10. It will be appreciated that air outlet 112 also serves to discharge any accumulated liquids that have resulted from respiratory activities, nasal emissions or perspiration. When platform 200 is attached to respiratory protective equipment (e.g., to mask 410 of FIG. 5A), the inferior location of air outlet 112 within the confined space (i.e., between the respiratory protective equipment and a face of a wearer) is conducive to the drainage of the gravitated accumulated liquid.

Communication device 114 is adapted to connect with external communication equipment such as a radio device (not shown) by socket 214, and may be firmly secured to main body 111 by fastening means 214a. Communication device 114 further comprises an internal speaker 214b positioned anteriorly to the mouth of the wearer of mask 10. The relative size of speaker 214b produces negligible interference to the incoming air through inlet 113.

Speech device 115 comprises a thin vibrating membrane 215 supported between perforated walls 215a and 215b, such as to allow the clear transmission of the wearer's voice and to prevent generation of undesirable noise.

FIGS. 2A-2C further show adjustable drinking straw 116 engaged with an inlet tube 216, having a drinking vessel connector 216a at its end. Connector 216a comprises lateral grasping protrusions 216b for comfortable grasping thereof. Protrusions 216b may also be utilized to press/release an internal valve for enabling/disabling the flow into tube 216. Connector 216a may utilize any suitable coupling arrangement, such as bayonet coupling 216c to connect with a corresponding arrangement of a drinking vessel (not shown).

Further shown in FIG. 2A are clamps 201 extending from main body 111 for securing cover 111b (FIG. 1) onto main body 111, and threading 202 for either threading lid 113a or an air filter into air inlet 113. However, one skilled in the art will readily realize alternative fastening and threading arrangements suitable for different applications and alternative interfaces that may be integrated within the inventive platform.

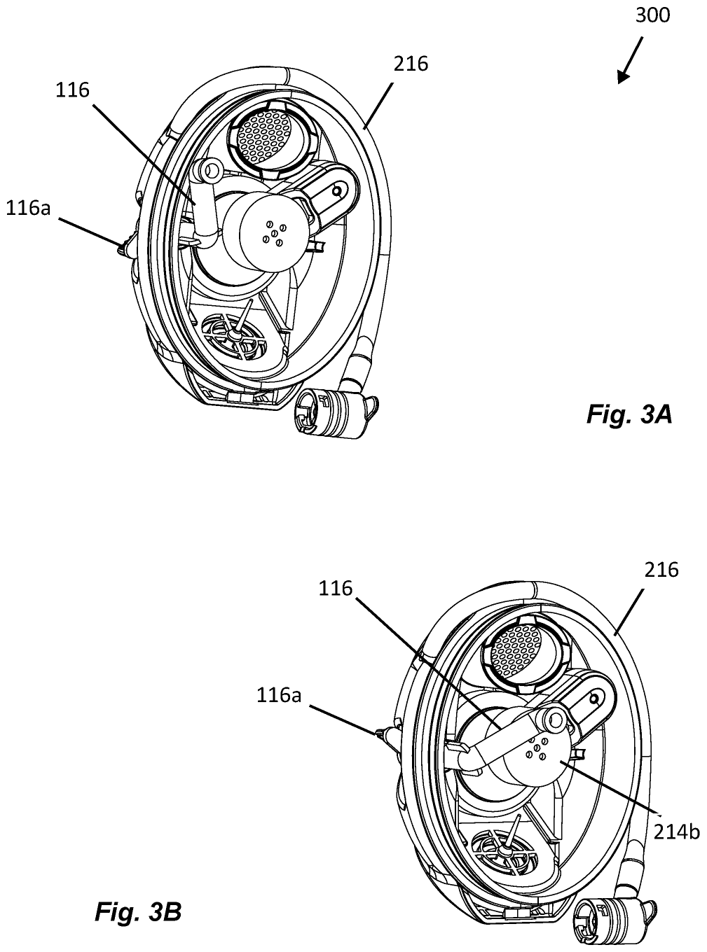

FIGS. 3A and 3B illustrate the two states of drinking straw 116, where FIG. 3A illustrates angled straw 116 in a stored state, in which adjustment lever 116a is in its default position and points towards the air outlet, and FIG. 3B illustrates straw 116 in a deployed state, in which adjustment lever 116a is in its adjusted position and points away from the air outlet. During manipulation of adjustment lever 116a, the anterior segment of angled straw 116, which is substantially parallel to direction of axial displacement of one-way valve 212 (FIG. 2C, rotates such that the free end of the posterior segment is set at a position that is accessible to the mouth of the wearer in the deployed state and at a position distant to the mouth of the wearer in the stored state. Shifting of knob 116a to a mediate position may enable the adjustment of straw 116 to accommodate differently shaped faces of wearers.

It can be readily noted that straw 116 is so positioned at both its stored and deployed states, such as to avoid interference between the various integrated interfaces. For example, straw 116 is mounted such that its posterior segment is positioned posteriorly to speaker 214b so as to avoid interference therebetween (best shown in FIG. 3B).

Also shown in 3A and 3B is the concavity of the main body. The increased surface area of the main body by virtue of such concavity is advantageously utilized to mount the various interfaces in compact and air-tight fashion as described hereinabove. The compact configuration of the platform main body by which the surface of a single cavity is utilized for significant space savings obviates the need of a plurality of separate cavities (i.e., one for each interface), thus eliminating potential void spaces in which CO2 is liable to accumulate. The inferior position of air outlet 112 within the single cavity of main body 111 facilitates continuous ventilation and evacuation of CO2.

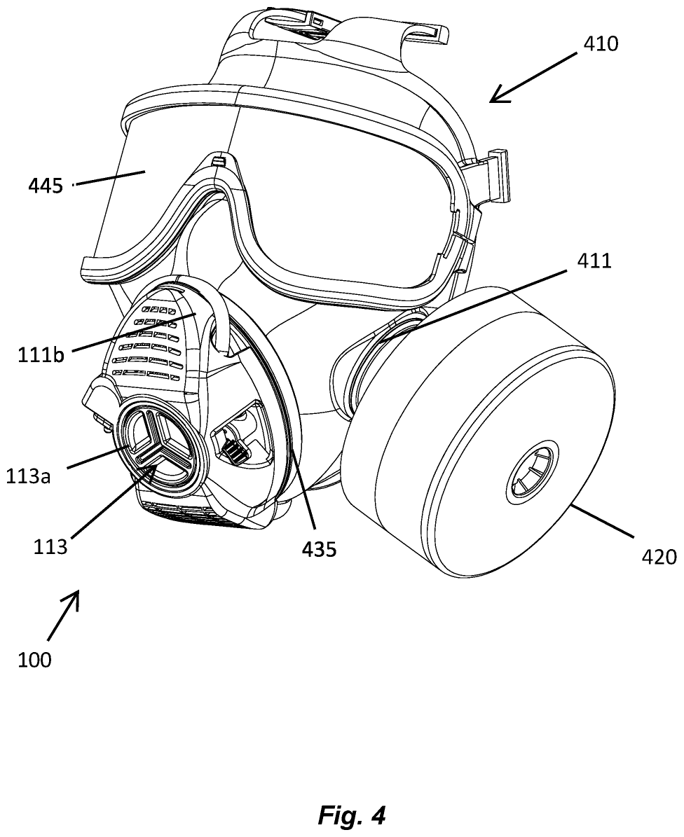

Of course, multiple different designs of mask 10 may be utilized with the inventive platform, such as the one shown in the following FIG. 4.

FIG. 4 shows a respiratory protective mask 410 with an integrated interface platform 100 and a conventional filter 420, according to an embodiment of the present invention. While mask 410 comprises a side air inlet 411, it can be noted that a second air filter (not shown) may be utilized in combination with or in lieu of filter 420, thereby providing an improved operational flexibility to the wearer of mask 410, further improving air capacity where needed. If the position of a filter is bothersome in any way, the filter may be simply removed from a first interface aperture and coupled to a second interface aperture while the first interface aperture is occluded to prevent the influx of noxious agents.

Also shown in FIG. 4 is an annular air-tight coupling 435 between platform 100 and mask 410 for maintaining sealed engagement therebetween.

Although not shown, a handle that is fixedly connected to the main body of platform 100 may be provided, in order to manipulate the platform when being coupled with mask 410. The handle may be superiorly positioned within platform 100 and be able to transmit a manual force applied by the user, when platform 100 is initially obliquely positioned and an inferior region of the posterior edge of the main body is in abutment with the edge surrounding the dedicated aperture 11 (FIG. 1) formed in mask 410. Thus a superior region of the posterior edge of the main body or of a round protrusion thereof will forcefully press on a sealing element provided with the surrounding edge of the aperture and the entire posterior edge of the main body or of a round protrusion thereof will be sealingly engaged with the edge surrounding the dedicated aperture. Alternatively, a grip provided with cover 111b may be manipulated to transmit the manual force applied by the user.

The handle or grip may also be manipulated when it is desired to disengage platform 100 from mask 410, for cleaning, maintenance or replacement purposes. If so desired, platform 100 may be used when separated from mask 410, for example the speech device may be interfaced with when platform 100 is separated from mask 410.

The versatility of mask 410 is reflected by not only the ability of platform 100 to be replaced, but also by the ability of goggles 445 to be sealingly replaced when it is desired, for example, to use a different type of safety goggles. The copending application by the same Applicant which bears attorney's docket number 43252/IL/21-ORP describes the apparatus for sealingly replacing safety goggles.

FIG. 5A shows a respiratory protective mask 500 with an integrated interface platform 100, according to an embodiment of the present invention. Mask 500 is provided with a nose-mouth cup 510 configured with a plurality of air guiding surfaces 511, which may laterally extend or otherwise protrude from the outer wall of nose-mouth cup 510, for ensuring efficient air flow and for reducing any void volume in which CO2 and humidity may accumulate. Nose-mouth cup 510 is coupled to platform 100 and serves to discharge the guided air through the outlet of platform 100.

FIG. 5B illustrates a posterior view of nose-mouth cup 510. Even though the nose and mouth of the wearer are protectively enclosed by cup 510, the wearer is able to access all of the integrated interfaces. To ensure suitable access of the various interfaces, an anterior surface is formed with dedicated apertures to receive the interfaces. The relative positioning and orientation of the integrated interfaces are shown with respect to posterior, peripherally extending resilient edge 512 of nose-mouth cup 510, which may be sealed and slightly inflated. Resilient edge 512 ensures a comfortable attachment of mask 500 with the wearer's face.

Although embodiments of the invention have been described by way of illustration, it will be understood that the invention may be carried out with many variations, modifications, and adaptations, without exceeding the scope of the claims.

1. An integrative interface platform for respiratory protective equipment, comprising a main body which is configured with at least five interfaces, with each of which a corresponding ancillary device, including an air inlet, is in fluid or sound communication with a confined space between said respiratory protective equipment and a face of a wearer, when said respiratory protective equipment is worn on a face, is sealingly engaged,
wherein second and third interfaces which are superiorly and inferiorly spaced, respectively, from a first interface are also angularly spaced in opposite directions from the first interface and the ancillary device of a fourth interface is posteriorly spaced from the ancillary device of the first interface.

2. The integrative interface platform according to claim 1, wherein the main body comprises five interfaces and each of the corresponding ancillary devices is selected from one or more of the group of:
a) an air outlet assembly, positioned at an inferior portion of the platform, which comprises a one-way valve for enabling evacuation of humid exhaled air containing CO2 via an air outlet during exhalation;
b) an air inlet assembly, which is located at an anterior region of the platform and in communication with a one-way valve for facilitating an influx of air via the air inlet during inhalation;
c) a communication device adapted to connect with external communication equipment;
d) a speech device, for enabling transmission therethrough of voice; and
e) an adjustable drinking implement.

3. The integrative interface platform according to claim 2, wherein the air outlet assembly is also configured to facilitate discharge of liquid that has accumulated at the inferior portion.

4. The integrative interface platform according to claim 2, wherein the air inlet is adapted to receive an outlet of a filtered air supply source.

5. The integrative interface platform according to claim 2, wherein the adjustable drinking implement is an adjustable drinking straw, comprising an adjustment lever which is positionable at a stored state or a deployed state, wherein said adjustable drinking straw connects with an inlet tube having a drinking vessel connector for enabling drinking therefrom.

6. The integrative interface platform according to claim 2, wherein the first interface is the air inlet, the second interface is an interface for the speech device, the third interface is the air outlet, the fourth interface is an interface for the communication device, and a fifth interface is an interface for the adjustable drinking implement.

7. Respiratory protective equipment, comprising the integrative interface platform according to claim 1.

8. The respiratory protective equipment according to claim 7, which is a mask.

9. The respiratory protective equipment according to claim 8, wherein the respiratory protective mask comprises one or more lateral air inlets, each of which is adapted to receive an outlet of a filtered air supply source.

10. The respiratory protective equipment according to claim 7, further comprising a hollow nose-mouth protective cup which is coupled to the platform while each of the corresponding ancillary devices remains in communication with the confined space.

11. The respiratory protective equipment according to claim 10, wherein the nose-mouth cup is configured with a plurality of air guiding surfaces which protrude from an outer wall of the nose-mouth cup to guide exhaled air through the air outlet of the platform.

12. The respiratory protective equipment according to claim 11, wherein the plurality of air guiding surfaces laterally extend from the outer wall of the nose-mouth cup.

13. The respiratory protective equipment according to claim 10, wherein a posterior edge of the nose-mouth cup is resilient.

14. The respiratory protective equipment according to claim 7, wherein the platform is sealingly engaged within an aperture formed in the respiratory protective equipment.

15. The respiratory protective equipment according to claim 14, further comprising an air-tight coupling between the platform and the respiratory protective equipment for maintaining sealed engagement therebetween.

16. The respiratory protective equipment according to claim 15, wherein the platform is disengageable from the respiratory protective equipment.

17. The respiratory protective equipment according to claim 15, wherein the air-tight coupling comprises a clamping tie that is engageable around an annular lip surrounding the aperture formed in the respiratory protective equipment and that is tightened by a fastening lock.

18. The respiratory protective equipment according to claim 15, wherein the platform comprises a handle by which a manual force that causes a posterior edge of the main body or of a protrusion thereof to forcefully press on a sealing element provided with a surrounding edge of the aperture formed in the respiratory protective equipment and to be sealingly engaged with the surrounding edge is transmittable.
